(19) United States
(12) Patent Application Publication
(10) Pub. No.: US 2023/0184917 A1
(43) Pub. Date: Jun. 15, 2023

(54) SYSTEM AND METHOD FOR VEHICLE INTERIOR DETECTION

(71) Applicants: Hyundai Motor Company, Seoul (KR); Kia Corporation, Seoul (KR)

(72) Inventors: Eung Hwan Kim, Seoul (KR); Seul Ki Jeon, Hwaseong (KR)

(21) Appl. No.: 17/888,033

(22) Filed: Aug. 15, 2022

(30) Foreign Application Priority Data
Dec. 13, 2021 (KR) 10-2021-0178095

(51) Int. Cl. G01S 13/50; B60R 11/00; A61B 5/113; A61B 5/11; B32B 17/06

(52) U.S. Cl. CPC G01S 13/50; B60R 11/00; A61B 5/113; A61B 5/1126; B32B 17/06; B60R 2011/0063; A61B 5/0816

(57) ABSTRACT

A system for vehicle interior detection, may include: a radar sensor having an emitting surface which emits a radar signal to a passenger compartment of a vehicle, and a plurality of conductive pins; a mounting bracket with which the radar sensor is mounted on a glass of the vehicle; a plurality of conductive layers which are provided on the glass; and a plurality of fixed contacts which are provided on the plurality of conductive layers, respectively, and which contact the plurality of conductive pins, respectively.

### CROSS-REFERENCE TO RELATED APPLICATION

This application claims under 35 U.S.C. § 119(e) the benefit of Korean Patent Application No. 10-2021-0178095, filed on Dec. 13, 2021, in the Korean Intellectual Property Office, the disclosure of which is incorporated herein in its entirety by reference.

### BACKGROUND

### Technical Field

The present disclosure relates to a system and a method for vehicle interior detection, and more particularly, to a system and a method for vehicle interior detection designed to accurately detect a person, an object, and/or the like inside/outside a passenger compartment using a radar sensor mounted in the passenger compartment of the vehicle.

### BACKGROUND

A vehicle includes a detection system designed to detect the presence/absence of an occupant in a passenger compartment, and to prevent a child from being left unattended in the passenger compartment and prevent an unauthorized person from approaching on or intruding into the vehicle.

A system for vehicle interior detection according to the related art includes a motion detection sensor (for example, an ultrasonic sensor) mounted on a room lamp of the vehicle. The motion detection sensor may be configured to detect the movement of a person in the passenger compartment of the vehicle.

In the system for vehicle interior detection according to the related art, however, the intensity/range of radio waves output from the motion detection sensor may be legally regulated considering the effect of radio waves on a human body, interference with the surroundings, and the like. Since the mounting position of the motion detection sensor may be relatively limited due to an interior structure of the vehicle, it may be difficult for one motion detection sensor to secure a sufficient detection area for the entire space of the passenger compartment. In order to solve this problem, a plurality of motion detection sensors may be mounted, which may increase costs and power consumption and make the system complicated.

The above information described in this background section may be provided to assist in understanding the background of the inventive concept, and may include any technical concept which may not be considered as the existing technologies that may be already known to those skilled in the art.

### SUMMARY

The present disclosure has been made to solve the above-mentioned problems occurring in the existing technologies while advantages achieved by the existing technologies may be maintained intact.

An embodiment of the present disclosure provides a system and a method for vehicle interior detection designed to accurately detect a person, a moving object, and/or the like inside/outside a passenger compartment using one or more radar sensors mounted in various positions of the vehicle.

According to an embodiment of the present disclosure, a system for vehicle interior detection may include: a radar sensor having a emitting surface which emits a radar signal to a passenger compartment of a vehicle, and a plurality of conductive pins; a mounting bracket with which the radar sensor may be mounted on a glass of the vehicle; a plurality of conductive layers which may be provided on the glass; and a plurality of fixed contacts which may be provided on the plurality of conductive layers, respectively, and which contact the plurality of conductive pins, respectively.

The mounting bracket may include a first bracket attached to the glass, and a second bracket detachably coupled to the first bracket. The first bracket and the second bracket may surround the radar sensor.

The first bracket may include an attachment wall attached to the glass through an attachment portion, and a skirt portion extending from the attachment wall. The attachment wall and the skirt portion may surround a portion of the radar sensor.

The attachment portion may include any one of an adhesive and a fastener.

The attachment wall may have an opening in which the plurality of conductive layers may be received.

The second bracket may be detachably fitted into the first bracket through a snap-fitting structure.

The second bracket may include a support wall supporting the radar sensor, and a skirt portion extending from the support wall. The support wall and the skirt portion may surround a portion of the radar sensor.

The support wall may have an opening, and the emitting surface of the radar sensor may be exposed to the passenger compartment through the opening of the support wall.

The snap-fitting structure may include a retention recess provided in the first bracket, and a retention barb provided on the second bracket. The retention barb may be detachably fitted into the retention recess.

The radar sensor may further include a frequency filter through which only a signal of a predetermined frequency band may be allowed to pass, and the frequency filter may remove a signal generated by an external impact.

The radar sensor may further include a thermal shield member preventing external heat from being transferred to the radar sensor.

According to another embodiment of the present disclosure, a method for vehicle interior detection may include: detecting, by a radar sensor, a moving object in a passenger compartment of a vehicle; and detecting, by the radar sensor, breathing and movement of a person when the moving object may be detected.

The method may further include removing, by a frequency filter, a signal generated by an external impact when the radar sensor receives the signal.

An exemplary embodiment of the system and a method for vehicle interior detection designed to accurately detect a person, a moving object, and/or the like inside/outside a passenger compartment described herein comprises using the system and method within the vehicle, where the described sensors are mounted in the vehicle or as a separate module configured to be added to a vehicle.

### BRIEF DESCRIPTION OF THE DRAWINGS

The above and other objects, features and advantages of the present disclosure will be more apparent from the following detailed description taken in conjunction with the accompanying drawings:

FIG. 1 illustrates a radar sensor of a system for vehicle interior detection according to an exemplary embodiment of the present disclosure, which may be mounted in a vehicle;

FIG. 2 illustrates a radar sensor of a system for vehicle interior detection according to an exemplary embodiment of the present disclosure, which may be mounted on a glass roof of a vehicle through a mounting bracket;

FIG. 3 illustrates a block diagram of a radar sensor of a system for vehicle interior detection according to an exemplary embodiment of the present disclosure;

FIG. 4 illustrates a flowchart of a method for vehicle interior detection according to an exemplary embodiment of the present disclosure;

FIG. 5 illustrates a graph of respiratory and motion signals which a radar sensor receives in a system for vehicle interior detection according to an exemplary embodiment of the present disclosure;

FIG. 6 illustrates a flowchart of a method for vehicle interior detection according to another exemplary embodiment of the present disclosure;

FIG. 7 illustrates a graph of a signal generated by a temporary impact which a radar sensor receives in a system for vehicle interior detection according to an exemplary embodiment of the present disclosure; and

FIG. 8 illustrates a graph of a signal generated by a continuous impact which a radar sensor receives in a system for vehicle interior detection according to an exemplary embodiment of the present disclosure.

### DETAILED DESCRIPTION

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

Hereinafter, exemplary embodiments of the present disclosure will be described in detail with reference to the accompanying drawings. In the drawings, the same reference numerals will be used throughout to designate the same or equivalent elements. In addition, a detailed description of well-known techniques associated with the present disclosure will be ruled out in order not to unnecessarily obscure the gist of the present disclosure.

Terms such as first, second, A, B, (a), and (b) may be used to describe the elements in exemplary embodiments of the present disclosure. These terms may be only used to distinguish one element from another element, and the intrinsic features, sequence or order, and the like of the corresponding elements may not be limited by the terms. Unless otherwise defined, all terms used herein, including technical or scientific terms, have the same meanings as those generally understood by those with ordinary knowledge in the field of art to which the present disclosure belongs. Such terms as those defined in a generally used dictionary may be to be interpreted as having meanings equal to the contextual meanings in the relevant field of art, and may not be to be interpreted as having ideal or excessively formal meanings unless clearly defined as having such in the present application.

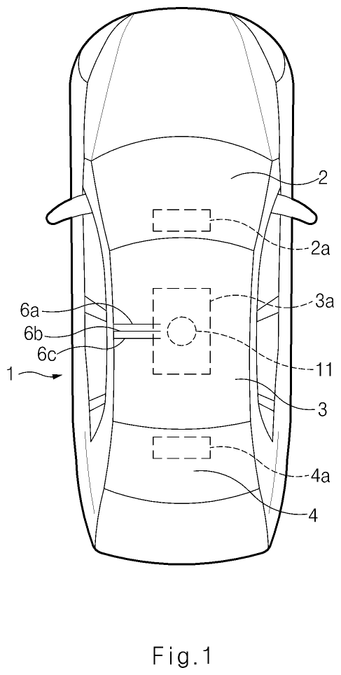

Referring to FIG. 1, a system for vehicle interior detection according to an exemplary embodiment of the present disclosure may include a radar sensor 11 selectively mounted in each of various positions 2, 3, and 4 in a passenger compartment of a vehicle 1.

The radar sensor 11 may be selectively mounted on at least one of a portion 2a of a front windshield 2 provided on the front of the vehicle 1, a portion 3a of a glass roof 3 provided on a roof of the vehicle 1, a portion 4a of a rear windshield 4 provided on the rear of the vehicle 1, and a side window glass provided on a side door of the vehicle 1. As the radar sensor 11 may be mounted on a translucent or transparent glass of the vehicle 1, a detection area/range of the radar sensor 11 may be relatively extended.

The radar sensor 11 may be configured to detect a human body or an object by detecting a change in radio-frequency (RF) transmission/reception. The radar sensor 11 may have various frequencies, and the radar sensor 11 may use various frequency ranges of millimeter waves and ultra wideband (UWB) (hundreds of MHz-GHz).

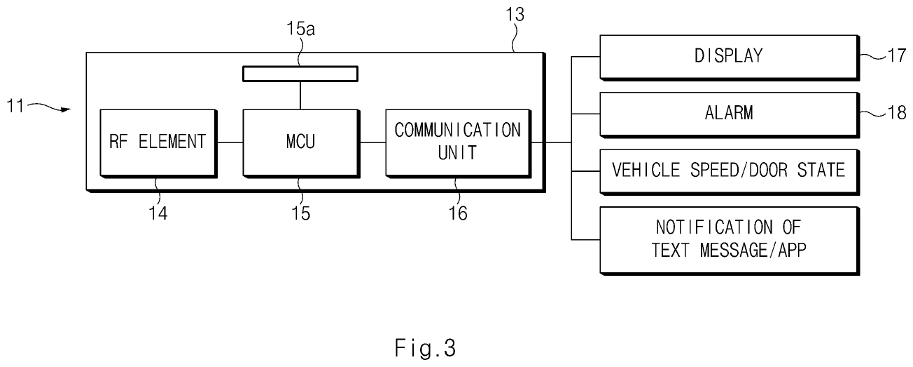

Referring to FIG. 3, the radar sensor 11 may include a sensor housing 13, and the sensor housing 13 may accommodate an RF module 14, a controller 15, and a communication unit 16.

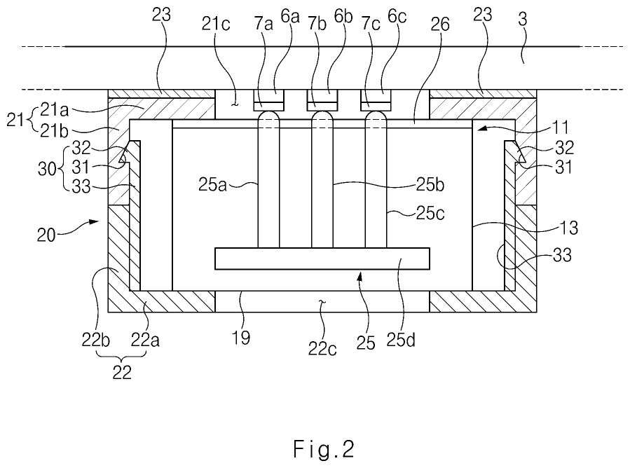

Referring to FIG. 2, the sensor housing 13 may include a first surface facing the glass of the vehicle such as the glass roof 3, and a second surface opposing the first surface. The second surface may face the passenger compartment of the vehicle. The sensor housing 13 may have a emitting surface 19 which emits a radar signal, and the emitting surface 19 of the radar sensor 11 may face the passenger compartment of the vehicle. The emitting surface 19 may be flush with the second surface of the sensor housing 13.

The RF module 14 may include an RF element and an antenna for transmitting and receiving the radar signal.

The controller 15 may be configured to process and apply the received radar signal. For example, the controller 15 may be a microcontroller unit (MCU).

The communication unit 16 may communicate with an external controller and an actuator through CAN communications, LIN communications, and the like. For example, the communication unit 16 may be connected to a display 17 provided on an instrument panel of the vehicle to communicate with the display 17, and the display 17 may display the operating state, detection result, and/or the like of the radar sensor 11. The communication unit 16 may be connected to an alarm 18 such as a lamp, a horn, an emergency light, or a buzzer provided on the instrument panel of the vehicle to communicate with the alarm 18, and the alarm 18 may provide visual and/or audible signals indicating the presence/absence of a person or a moving object in the passenger compartment of the vehicle through. The communication unit 16 may receive information on the speed of the vehicle, the open/closed state of a vehicle door, and the like from a vehicle controller. The communication unit 16 may notify a user/a driver of the presence/absence of a person or a moving object in the passenger compartment through a text message or an app on the user's terminal such as the driver's mobile phone/smart phone.

Referring to FIG. 2, the system for vehicle interior detection according to an exemplary embodiment of the present disclosure may include a mounting bracket 20 with which the radar sensor 11 may be mounted in the vehicle 1.

According to an exemplary embodiment, the radar sensor 11 may be mounted on the glass roof 3 through the mounting bracket 20. That is, the mounting bracket 20 is configured to secure the radar sensor 11 on the glass roof 3. As the radar sensor 11 may be mounted on the glass roof 3, the radar sensor 11 may more extensively and accurately detect the presence/absence of a person or a moving object in the interior of the vehicle (that is, the passenger compartment).

Referring to FIG. 2, a plurality of conductive layers 6a, 6b, and 6c for power transmission and signal transmission may be provided on the glass roof 3. A flexible wire may be attached to the glass roof 3 through adhesive or a conductive ink may be applied to the glass roof 3 so that the conductive layers 6a, 6b, and 6c of a predetermined pattern may be provided on the glass roof 3. A plurality of fixed contacts 7a, 7b, and 7c may be provided on the plurality of conductive layers 6a, 6b, and 6c, respectively.

Referring to FIG. 2, the radar sensor 11 may be firmly mounted on the glass of the vehicle such as the glass roof 3 through the mounting bracket 20. The mounting bracket 20 may include a first bracket 21 attached to the glass, and a second bracket 22 detachably coupled to the first bracket 21.

The first bracket 21 may surround a portion of the sensor housing 13 of the radar sensor 11, and the first bracket 21 may be attached to the glass of the vehicle such as the glass roof 3 through an attachment portion 23 such as an adhesive and a fastener. The adhesive may be a glue, a double-sided sticker, and/or the like, and the fastener may be a screw, a rivet, and/or the like. The first bracket 21 may include an attachment wall 21a, and a skirt portion 21b extending from the attachment wall 21a. At least a portion of the first surface of the sensor housing 13 may contact the attachment wall 21a. The attachment wall 21a may be attached to the glass of the vehicle such as the glass roof 3 through the attachment portion 23. The attachment wall 21a may have an opening 21c, and the conductive layers 6a, 6b, and 6c may be received in the opening 21c. The attachment wall 21a and the skirt portion 21b may partially surround the sensor housing 13.

The second bracket 22 may surround a remaining portion of the sensor housing 13 of the radar sensor 11, and the second bracket 22 may be detachably coupled to the first bracket 21 through a snap-fitting structure 30. The second bracket 22 may include a support wall 22a, and a skirt portion 22b extending from the support wall 22a. The support wall 22a may support at least a portion of the second surface of the sensor housing 13. The support wall 22a may have an opening 22c. The emitting surface 19 of the sensor housing 13 of the radar sensor 11 may be exposed to the passenger compartment through the opening 22c, and accordingly the radar signal may be widely radiated to the passenger compartment through the emitting surface 19.

The snap-fitting structure 30 may include a retention recess 31 provided in the first bracket 21, and a retention barb 32 provided on the second bracket 22. The retention recess 31 may be provided in the skirt portion 21b of the first bracket 21, and a retention arm 33 may extend from the second bracket 22 toward the skirt portion 21b of the first bracket 21, and the retention barb 32 may be provided on a free end of the retention arm 33. The retention barb 32 may be detachably fitted into the retention recess 31 so that the second bracket 22 may be detachably coupled to the first bracket 21.

As described above, the first bracket 21 and the second bracket 22 may be coupled through the snap-fitting structure 30 so that the radar sensor 11 may be firmly attached to the glass of the vehicle such as the glass roof 3.

Referring to FIG. 2, the sensor housing 13 of the radar sensor 11 may be stably supported to the attachment wall 21a of the first bracket 21 and the support wall 22a of the second bracket 22, and the emitting surface 19 of the sensor housing 13 may be exposed to the passenger compartment through the opening 22c.

The radar sensor 11 may include a connector 25 provided on an exterior surface of the sensor housing 13, and the connector 25 may include a connector housing 25d, and a plurality of conductive pins 25a, 25b, and 25c extending from the connector housing 25d. The conductive pins 25a, 25b, and 25c may contact the fixed contacts 7a, 7b, and 7c of the glass roof 3, respectively, and the radar sensor 11 may be electrically connected to the conductive layers 6a, 6b, and 6c of the glass roof 3. The plurality of conductive pins 25a, 25b, and 25c may be of VCC, GND, CAN high, CAN low, LIN signal, Ethernet, and/or the like. Each of the conductive pins 25a, 25b, and 25c may be elastically supported by an elastic member such as a spring. Since the radar sensor 11 may be mounted on the glass such as glass roof 3 through the conductive layers 6a, 6b, 6c and the conductive pins 25a, 25b, 25c, interruption by interior component (e. g. vehicle seat) or sunroof may be minimized so that the detection accuracy of the radar sensor 11 may be improved.

The radar sensor 11 may further include a frequency filter 15a through which only a signal of a predetermined frequency band may be allowed to pass. For example, the frequency filter 15a may be a low-pass filter, a band-pass filter, or the like. The frequency filter 15a may be a hardware module electrically connected to the controller 15 or a software module embedded in the controller 15.

Since the radar sensor 11 may be mounted on the glass of the vehicle such as the front windshield 2, the glass roof 3, and the rear windshield 4, an external impact caused by rain, hail, a falling object, and/or the like may be applied to the radar sensor 11. When the external impact may be applied to the radar sensor 11, the detection accuracy of the radar sensor 11 may be relatively reduced due to a signal (noise) generated by the external impact. To solve this problem, the frequency filter 15a may remove the signal (noise) generated by the external impact, and the radar sensor 11 may only receive a signal of a frequency band set by the frequency filter 15a, and thus the radar sensor 11 may detect the person or the moving object in the passenger compartment more accurately.

When direct sunlight may be transmitted to the radar sensor 11 through the glass, the radar sensor 11 may be heated by external heat, and thus the external heat may cause signal distortion and/or sensor performance failure. In order to solve the external heat problem, the radar sensor 11 may be configured to compensate for signal distortion caused by the external heat when an indoor temperature measured by a temperature sensor provided in the radar sensor 11 may be increased to a threshold value or higher.

Referring to FIG. 2, the radar sensor 11 may further include a thermal shield member 26 attached to the first surface of the sensor housing 13. The thermal shield member 26 may prevent the external heat from being transferred to the sensor housing 13 of the radar sensor 11. The thermal shield member 26 may be at least one of a thermal shield film, a thermal shield gel, and a thermal absorber.

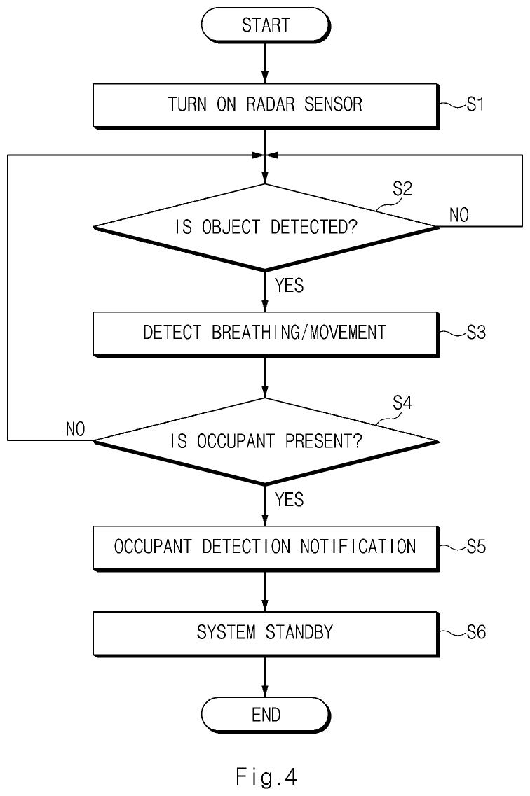

FIG. 4 illustrates a flowchart of a method for vehicle interior detection according to an exemplary embodiment of the present disclosure.

Referring to FIG. 4, after the radar sensor 11 may be turned on (51), the radar sensor 11 may sense a change between a transmission signal and a reception signal to thereby detect the presence/absence of a moving object in the passenger compartment of the vehicle (in particular, in a detection area of the radar sensor 11) (S2).

When the moving object is detected in the passenger compartment of the vehicle in S2, the radar sensor 11 may detect the breathing or movement of a person (S3). A signal that the radar sensor 11 transmits may be reflected by the person's breathing and movement, and the radar sensor 11 may receive the reflected signal. Based on a difference between the signal transmitted by the radar sensor 11 and the signal received by the radar sensor 11, a distance between the radar sensor 11 and the subject (person, object, etc.), and the movement (direction, strength, etc.) of the subject may be determined.

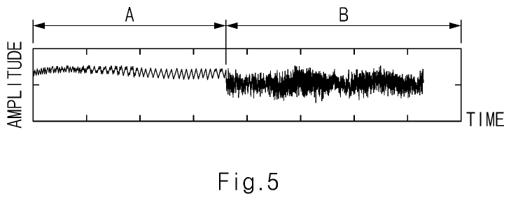

For example, like area A shown in FIG. 5, the person's breathing may be regular and be indicated by a signal of relatively low amplitude, and a respiratory rate may be determined by converting its repeatability in minutes. Like area B shown in FIG. 5, the person's movement may be less regular, and the radar sensor 11 may receive a signal of relatively high amplitude in proportion to the motion magnitude. That is, the amplitude of the reception signal may increase in accordance with the person's motion magnitude.

As the radar sensor 11 detects the breathing and movement of the person, the controller 15 may determine whether an occupant is present in the passenger compartment of the vehicle (S4).

When it is determined that the occupant is present in the passenger compartment, the controller 15 may transmit a notification signal indicating the presence of the occupant to the display 17 and the alarm 18 of the vehicle and the user's terminal (S5), and the system for vehicle interior detection may be in standby mode (S6).

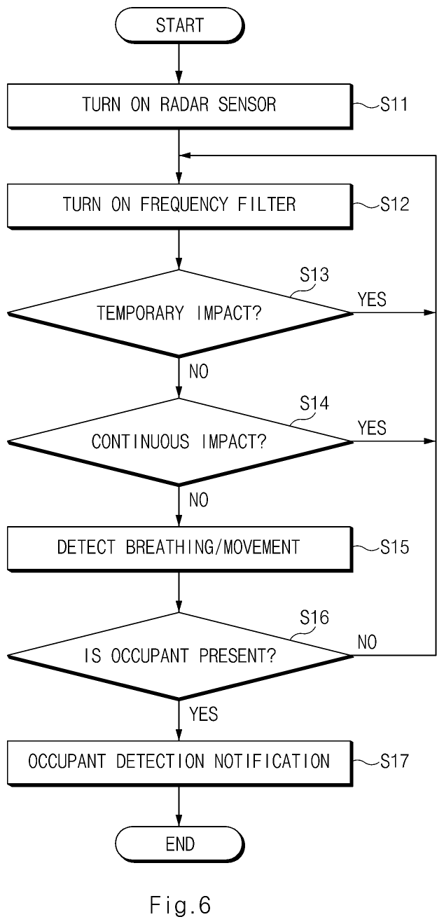

FIG. 6 illustrates a flowchart of a method for vehicle interior detection according to another exemplary embodiment of the present disclosure.

Referring to FIG. 6, after the radar sensor 11 may be turned on (S11), the frequency filter 15a may be turned on (S12).

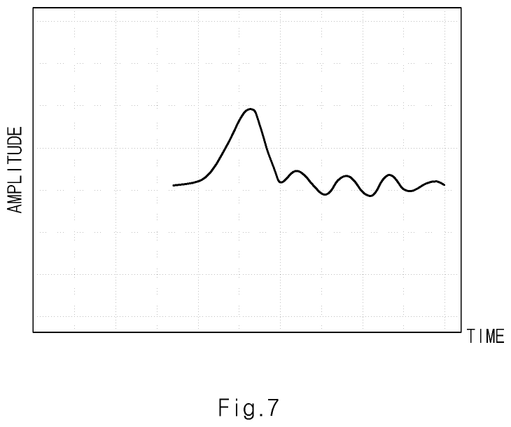

After the frequency filter 15a is turned on, it may be determined whether the radar sensor 11 receives a signal generated by a temporary impact as a falling object temporarily falls onto the vehicle 1 (S13). When it is determined in S13 that the radar sensor 11 receives the signal generated by the temporary impact, the frequency filter 15a may remove the signal generated by the temporary impact. When the falling object temporarily falls onto the vehicle 1, the radar sensor 11 may receive the signal (see FIG. 7) according to the temporary impact.

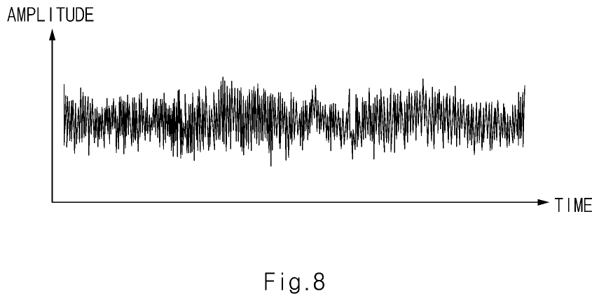

When it is determined in S13 that the radar sensor 11 does not receive any signal caused by the temporary impact as the falling object does not temporarily fall onto the vehicle 1, it may be determined whether the radar sensor 11 receives a signal generated by a continuous impact as the rain, hail, or the like continuously falls vertically onto the vehicle 1 (S14). When it is determined in S14 that the radar sensor 11 receives the signal generated by the continuous impact, the frequency filter 15a may remove the signal generated by the continuous impact. When the rain, hail, or the like repeatedly and continuously falls onto the vehicle 1, the radar sensor 11 may receive the signal (see FIG. 8) according to the continuous impact.

When it is determined in S14 that the radar sensor 11 does not receive any signal caused by the continuous impact as the rain, hail, or the like does not continuously fall onto the vehicle 1, the radar sensor 11 may detect the breathing or movement of a person (S15). A signal that the radar sensor 11 transmits may be reflected by the person's breathing and movement, and the radar sensor 11 may receive the reflected signal. Based on a difference between the signal transmitted by the radar sensor 11 and the signal received by the radar sensor 11, a distance between the radar sensor 11 and the subject, and the movement (direction, strength, etc.) of the subject may be determined.

For example, like area A shown in FIG. 5, the person's breathing may be regular and be indicated by a signal of relatively low amplitude, and a respiratory rate may be determined by converting its repeatability in minutes. Like area B shown in FIG. 5, the person's movement may be less regular, and the radar sensor 11 may receive a signal of relatively high amplitude in proportion to the motion magnitude. That is, the amplitude of the reception signal may increase in accordance with the person's motion magnitude.

As the radar sensor 11 detects the breathing and movement of the person, the controller 15 may determine whether an occupant is present in the passenger compartment of the vehicle (S16).

When it is determined that the occupant is present in the passenger compartment, the controller 15 may transmit a notification signal indicating the presence of the occupant to the display 17 and the alarm 18 of the vehicle and the user's terminal (S17).

As set forth above, the system and the method for vehicle interior detection according to exemplary embodiments of the present disclosure may be designed to accurately detect a person, an object, and/or the like inside/outside the passenger compartment using the radar sensor mounted on the glass of the vehicle, thereby preventing a child from being left unattended in the passenger compartment, providing information on the number and locations of occupants in the passenger compartment, transmitting information about the occupants in the event of an accident, and providing a notification to remind the occupants to wear seatbelts. In addition, they may be designed to prevent and warn the intrusion of an unauthorized person into the vehicle.

Hereinabove, although the present disclosure has been described with reference to exemplary embodiments and the accompanying drawings, the present disclosure may not be limited thereto, but may be variously modified and altered by those skilled in the art to which the present disclosure pertains without departing from the spirit and scope of the present disclosure claimed in the following claims.

What is claimed is:

1. A system for vehicle interior detection of a vehicle, the system comprising:
a radar sensor having a emitting surface configured to emit a radar signal to a passenger compartment of a vehicle, and a plurality of conductive pins;
a mounting bracket configured for the radar sensor to mount on a glass of the vehicle;
a plurality of conductive layers configured to be provided on the glass; and
a plurality of fixed contacts which are provided on the plurality of conductive layers, respectively, and which contact the plurality of conductive pins, respectively.

2. The system according to claim 1, wherein the mounting bracket includes a first bracket configured to be attached to the glass, and a second bracket detachably coupled to the first bracket, and
the first bracket and the second bracket surround the radar sensor.

3. The system according to claim 2, wherein the first bracket includes an attachment wall configured to be attached to the glass through an attachment portion, and a skirt portion extending from the attachment wall, and
the attachment wall and the skirt portion surround a portion of the radar sensor.

4. The system according to claim 3, wherein the attachment portion includes any one of an adhesive and a fastener.

5. The system according to claim 3, wherein the attachment wall has an opening in which the plurality of conductive layers are received.

6. The system according to claim 2, wherein the second bracket is detachably fitted into the first bracket through a snap-fitting structure.

7. The system according to claim 6, wherein the second bracket includes a support wall supporting the radar sensor, and a skirt portion extending from the support wall, and
the support wall and the skirt portion surround a portion of the radar sensor.

8. The system according to claim 7, wherein the support wall has an opening, and
the emitting surface of the radar sensor is exposed to the passenger compartment through the opening of the support wall.

9. The system according to claim 6, wherein the snap-fitting structure includes a retention recess provided in the first bracket, and a retention barb provided on the second bracket, and
the retention barb is detachably fitted into the retention recess.

10. The system according to claim 1, wherein the radar sensor further includes a frequency filter through which only a signal of a predetermined frequency band is allowed to pass, and
the frequency filter removes a signal generated by an external impact.

11. The system according to claim 1, wherein the radar sensor further includes a thermal shield member preventing external heat from being transferred to the radar sensor.

12. The system according to claim 1, comprising the vehicle.

13. A method for vehicle interior detection, the method comprising:
detecting, by a radar sensor, a moving object in a passenger compartment of a vehicle; and
detecting, by the radar sensor, breathing and movement of a person when the moving object is detected.

14. The method according to claim 13, further comprising removing, by a frequency filter, a signal generated by an external impact when the radar sensor receives the signal.
